(19) United States
(12) Patent Application Publication
(10) Pub. No.: US 2011/0060206 A1
(43) Pub. Date: Mar. 10, 2011

(54) Device for determining body functions

(76) Inventors: Ulrich Schaaf, Kaisersbach (DE); Andreas Kugler, Alfdorf (DE); Ulrich Ladstaetter, Weinstadt (DE); Patrick Stihler, Ostfildern (DE)

(21) Appl. No.: 12/806,310

(22) Filed: Aug. 9, 2010

(30) Foreign Application Priority Data
Sep. 4, 2009 (DE) 10 2009 029 215.2

(51) Int. Cl. A61B 5/04; G08B 7/06

(52) U.S. Cl. 600/372; 340/573.1

(57) ABSTRACT

A device for determining one or multiple body functions of a person includes at least one body function sensor, an electronic unit, and a fixing arrangement for detachably fastening the device to the body of the person. The electronic unit includes a first film having a printed conductor structure, at least one electronic component situated on the first film and contacting the printed conductor structure, and a second film. To encapsulate the electronic component, the second film is laminated onto the side of the first film on which the electronic component is situated.

### BACKGROUND OF THE INVENTION

1. Field of the Invention

The present invention relates to a device and method for determining one or multiple body functions of a person.

2. Description of Related Art

Published German utility model application DE 203 01 410 U1 describes a patch having electrodes and an electronics system for detecting, storing, and transmitting vital parameters. However, the encapsulation of the electronics system is relatively complicated.

### BRIEF SUMMARY OF THE INVENTION

The present invention provides a device for determining one or multiple body functions of a person, including at least one body function sensor, one electronic unit, and one fixing means for detachably fastening the device to the body of the person. The electronic unit includes a first film having a printed conductor structure, at least one electronic component situated on the first film and contacting the printed conductor structure, and a second film, the second film being laminated onto the side of the first film on which the electronic component is situated.

An electronic unit having this type of design has the advantage that the electronic component may be encapsulated by laminating the second film thereon. The reliability of the electronic component may be advantageously increased in this way. In addition, the use of films allows a flexible electronic unit to be achieved which, for example, may be adapted to the shape and contour of the person's body. This in turn may have an advantageous effect on the wearing comfort for the person. Furthermore, as the result of such a design the usable surface of the electronic unit may be increased, or the electronic unit may have a relatively flat design.

In one example embodiment of the present invention, a dielectric material, in particular a dielectric layer, is situated between the first and the second film. For example, the dielectric material may be situated between and on the electronic components.

In another example embodiment of the present invention, an insulating layer is applied to at least one outwardly facing side of the electronic unit. In this way, the electronic unit may also be protected from environmental influences. An insulating layer is preferably applied on both outwardly facing main surfaces of the electronic unit. “Main surfaces of the electronic unit” refers in particular to the largest surfaces of the electronic unit situated opposite to one another. This has the advantage on the one hand that the electronic unit may be better protected from environmental influences. On the other hand, an electronic unit having such a design may be shaped using an embossing process and/or a deep-drawing process in such a way that the electronic unit is completely encapsulated by an insulating layer. This in turn may have a further advantageous effect on protecting the electronic unit from environmental influences.

The device preferably determines one or multiple body functions selected from the group composed of heartbeat, in particular of the fetus, respiration, for example respiratory flow (heat flow from oral and nasal breathing), body temperature, pulse, motion of a body part, for example an arm, leg, or the head, and electric body currents, for example during electrocardiography (EKG), in particular during cordless electrocardiography (EKG).

In particular, the device may thus include one or multiple body function sensors selected from the group composed of motion sensors, temperature sensors, blood oxygen sensors, noise sensors, and electricity sensors.

In another example embodiment of the present invention, the device thus includes an output device for cordlessly outputting data. This has the advantage that the person does not have to be connected to a data processing system via a data cable for the duration of the measurement. In this way it is advantageously not necessary to restrict the mobility of the person using bulky equipment and/or to hinder sleeping, which may increase the comfort of the person. The output device preferably has a design that is flexible and/or bendable and/or adjustable in its shape.

The data determined by the body function sensor may then be made directly visible with the aid of an additionally integrated visual method, for example on the basis of color indication or a flexible LCD display. This may represent a very simple method for displaying the occurrence of an event.

In another example embodiment of the present invention, the output device is a display device, for example an LED, an LED combination, an OLED, an OLED combination, an LCD display, a TLC display, a display based on an electrochemical color reaction, or an electrophoretic display. Ascertained data or results may thus be advantageously displayed directly on the device via an optical imaging method. The output device in particular may be a display device which displays body function characteristic values, for example the quantity and/or the potential, via a display, for example a text display and/or a color code, for example using differently colored LED/OLEDs, for example based on a traffic light display of the colors green, yellow, and red, and/or a color change (occurrence or non-occurrence of an event). On the basis of this display the person, for example by using an operating manual, may read out an indicated bodily state, in particular the state of health or the state of training.

The data determined by the body function sensor may also be transmitted by active transmission, in particular contactless and/or cordless transmission, for example wireless radio transmission, to a receiving device, and may optionally be evaluated by an evaluation device connected to the receiving device or integrated therein.

In another example embodiment of the present invention, the output device is thus an active transmitter for wirelessly transmitting data, for example an infrared transmitter or a radio transmitter, in particular a WLAN transmitter or a Bluetooth transmitter. The output device may be designed in particular for transmitting data to a separate receiving device. The device according to the present invention may thus be used over a long period of time.

It is likewise possible that the data determined by the body function sensor, e.g., contactless and/or cordless sensor using RFID technology via a printed antenna, are read out by a receiving device and may optionally be evaluated by an evaluation device connected to the receiving device or integrated therein.

In another example embodiment of the present invention, the output device is therefore a passive transmitter for wirelessly transmitting data, for example an RFID transponder.

The output device may also be designed for transmitting data to a separate receiving device. This likewise has the advantage that the device according to the present invention may be used over a long period of time.

In another example embodiment of the present invention the device includes a power supply device. The power supply device is preferably designed for supplying power to the body function sensor(s) and/or the electronic unit and/or the output device and/or a memory device and/or an evaluation device. The power supply device may be an autonomous and/or cordless power supply device. An “autonomous power supply device” refers in particular to a power supply device which does not have to be connected to the power network for voltage supply. For example, the power supply device may be a battery or an accumulator, in particular a battery.

The device according to the present invention is preferably cordless. “Cordless” is understood in particular to mean that the device may be operated without a cable connection to another device.

In another example embodiment of the present invention the device includes an evaluation device, in particular for evaluating raw data of the body function sensor. The evaluation device may be based, for example, on an integrated circuit (IC), in particular a logical integrated circuit.

In another example embodiment of the present invention the device includes a memory device, in particular for storing data, in particular raw data which are output by the body function sensor, or data at least partially evaluated by the evaluation device, for example. In this way, simultaneous monitoring/data readout is not always necessary, and instead the measurement of the body function may be made over a fairly long period of time, for example eight hours, in particular for indicating body functions during sleep.

The device may also have a combined memory and evaluation device.

In another example embodiment of the present invention, the body function sensor and/or the output device and/or the power supply device and/or the evaluation device and/or the memory device are partially or completely integrated into the electronic unit. For example, the electronic components of the body function sensor and/or of the output device in particular may be integrated into the electronic unit, electrical components such as electrodes and/or antennas being situated outside the electronic unit. However, the body function sensor and/or the output device may also be completely integrated into the electronic unit. The power supply device and/or the evaluation device and/or the memory device are preferably completely integrated into the electronic unit.

In another example embodiment of the present invention, the body function sensor and/or the output device and/or the power supply device and/or the evaluation device and/or the memory device are electronic components of the electronic unit. In particular, the electronic components of the electronic unit may be selected from the group composed of integrated circuits (IC), sensors, power supply devices, in particular batteries, output devices, in particular active or passive transmitters, for example RFID transponders, receiving devices, in particular antennas, evaluation devices, and memory devices.

The fixing means is preferably detachably fastenable to a finger, a toe, an ear, in particular an earlobe, the neck, a temple, or the forehead of a person.

In another example embodiment of the present invention the fixing means is designed as a patch, for example a single-use patch or a multi-use patch, in particular a single-use patch. For example, the patch may have a carrier layer, for example made of cellulose or woven fabric, and an adhesive layer, for example based on acrylate, situated thereon. In particular, the body function sensor and/or the electronic unit may be integrated into a medical patch and/or a homeopathic patch and/or an athletic patch, which is also used as a fixing means. For example, the patch may have a cover layer which faces the adhesive layer and is laminated onto the carrier layer, and the body function sensor and/or the electronic unit is affixed to the carrier layer and is covered, for example to avoid sticking thereto.

In another example embodiment of the present invention the device is designed as a sensor patch. For example, by affixing such a patch, body functions such as heartbeat (also heart sounds during pregnancy), respiration, body temperature, pulse, motion of a body part, and electric body currents, may be detected, for example during a defined period of time. A further advantage is the comfortable measurement and monitoring of body function data at any desired time.

The electronic unit may be manufactured using, for example, a method for producing an electronic module which includes at least one electronic component, and a printed conductor structure with which the at least one electronic component is contacted, the method including the following steps:
structuring an electrically conductive layer of a first film for forming the printed conductor structure;
fitting the printed conductor structure with the at least one electronic component; and
laminating a second film onto the first film fitted with the electronic component, on the side on which the first film is fitted with the electronic component.

The device for determining one or multiple body functions of a person may then be produced by mounting the electronic unit and/or a body function sensor on a fixing means, in particular by integrating the electronic unit and/or a body function sensor into a patch, for example using a laminating process.

The second film may likewise have a printed conductor structure and at least one electronic component, the at least one electronic component of the second film being situated on the second film and contacting the printed conductor structure of the second film. This may be achieved, for example, by structuring an electrically conductive layer of a second film for forming the printed conductor structure, and fitting the printed conductor structure of the second film with at least one electronic component. The lamination is preferably carried out in such a way that the electronic components of the first and second films face one another, so that the printed conductor structures are situated on the outer sides of the electronic unit. The surface area utilization may thus be greatly increased.

The printed conductor structures of the first and/or second film may be situated opposite from the electronic component provided on the first and/or second film.

The first and/or second film may in particular include an electrically conductive layer and an electrically nonconductive layer, the electrically conductive layer being structured for forming the printed conductor structure, and the electrically nonconductive layer being unstructured and used as a carrier layer.

The electrically conductive layers, i.e., the printed conductor structures, may independently be composed of a metal or a metal alloy, for example copper, silver, gold, platinum, or palladium, or an alloy thereof.

It is possible to deform the electronic unit when a first and/or second film made of a thermoplastic material is used. Use of a thermoplastic material has also proven to be advantageous when the electronic unit is completely encapsulated using a final embossing process and/or deep-drawing process.

To prevent unwanted electrical connections which result from laminating the second film, a dielectric material, for example a dielectric plastic, in particular a dielectric thermoplastic plastic, is preferably provided between the first and the second film. In addition, the dielectric material preferably fixes the electronic components.

To introduce the dielectric material between the first and the second layer, it is possible, for example, to apply a coating made of the dielectric material to the first film before fitting with the electronic component, it being possible to apply the coating made of the dielectric material before or after the electrically conductive layer is structured to form the printed conductor structure. Applying the coating before structuring has the advantage that the dielectric layer forms a carrier layer on which the metal layer may be structured.

However, it is alternatively possible, for example, for the dielectric material to be applied to the side of the first or second film on which the at least one electronic component is positioned, after the first and/or second film has been fitted. It is also possible to apply a further dielectric layer to the first or second film after the first and/or second film has been fitted. A coating which has been applied before the first or second film is fitted may be situated on the side on which the first or second film has been fitted with the electronic component, or on the side facing away from the electronic component.

The printed conductor structure of the first film and the printed conductor structure of the second film may be connected to one another via one or more feed-throughs. Feed-throughs may be introduced, for example, by providing boreholes and then metal-coating same. Alternatively, the feed-throughs may be produced, for example, using a deep-drawing process in which the upper layer of a conductive film is pressed over a mandrel through the dielectric material, until the dielectric material contacts the lower conductive film.

An insulating layer is preferably applied to at least one outwardly facing layer of the electronic unit. The insulating layer is preferably likewise made of a dielectric material, for example a dielectric plastic, in particular a thermoplastic dielectric plastic. In this case, using a thermoplastic plastic may also be advantageous when the electronic unit is ultimately to be deformed or completely encapsulated using an embossing process and/or a deep-drawing process.

In addition, the first film, the second film, the dielectric layer, and/or the insulating layer may be made of an elastomer, for example a ductile silicone. By using elastomers in conjunction with suitable printed conductor structures, electronic circuits may be produced which are flexible in multiple spatial directions. In particular, such electronic units may also be stretched, for example.

At least two electronic units may be joined together to form an electronic composite unit. For this purpose, for example, multiple first films may be laminated on each other, each having, for example, at least one electronic component on each side. The individual electronic units may be connected to one another via feed-throughs. For an electronic composite unit, a dielectric material may be provided between two first films in each case. The dielectric material may be a plastic or a silicone, for example.

To achieve improved encapsulation of the electronic unit, the electronic component or electronic components may be enclosed by a molding compound. Additional stabilization of the electronic component and of the contact, for example, may be achieved in this way, so that the electronic component does not break when the electronic module is flexed. At least one electronic component situated on the first and/or second film is preferably enclosed by a molding compound.

The electronic unit may be shaped into a molded part in a final step. The shaping may be carried out, for example, by embossing and/or deep drawing or other shaping processes. To prevent electronic components with which the film(s) is/are fitted from being damaged during the shaping process, it is possible on the one hand to carry out the fitting in such a way that no electronic components are mounted at bending or kink points of the molded part. On the other hand, it is also possible, for example, to use flexible electronic components which may be bent, for example.

The electronic components may be mounted on the first and/or second film with the aid of flip-chip technology. The electronic components may be provided with contact points (bumps), for example. The contact points may be pressed through the electrically nonconductive layer of the first and/or second film so that the contact points contact the printed conductor structure situated therebeneath.

The present invention also provides a method for determining one or multiple body functions of a person, using a device according to the present invention in which at least one body function sensor measures one body function, and the output device outputs data which are a function of the measured body function. An evaluation device of the device according to the present invention is able in particular to evaluate the raw data of the body function sensor with regard to body function characteristic values, for example the quantity and/or the potential of the body function. The output device may then directly display body function characteristic values or output same to a receiving device. In addition, the memory device may store the raw data of the body function sensor, and after a defined period of time the evaluation device retrieves the raw data stored in the memory device and evaluates these data with regard to body function characteristic values. Furthermore, a receiving device may receive the data which are output by the output device. For example, the receiving device may read out the data which are output by the output device. The receiving device may actively transmit a signal. For example, the receiving device according to the present invention may generate a high-frequency alternating electromagnetic field to activate the passive transmitter of the output device for wirelessly transmitting data. In addition, the memory device may first store the raw data of the body function sensor, and the output device may then output the raw data based on the body function to the particularly separate receiving device. The raw data may then be evaluated by the evaluation device of the receiving device with regard to body function characteristic values, and displayed by a display device of the receiving device.

A further subject matter of the present invention is the use of a device according to the present invention, in particular as a single-use patch and/or in the field of medical technology, for example telemedicine, for example for cordless determination of the heartbeat, in particular of the fetus, the respiration, for example the respiratory flow (heat flow from oral and nasal breathing), the body temperature, the pulse, the motion of a body part, for example an arm, leg, or the head, and electric body currents, for example during electrocardiography (EKG).

### BRIEF DESCRIPTION OF THE DRAWING

FIG. 1 shows a schematic cross section of an example embodiment of a device according to the present invention, designed as a sensor patch.

### DETAILED DESCRIPTION OF THE INVENTION

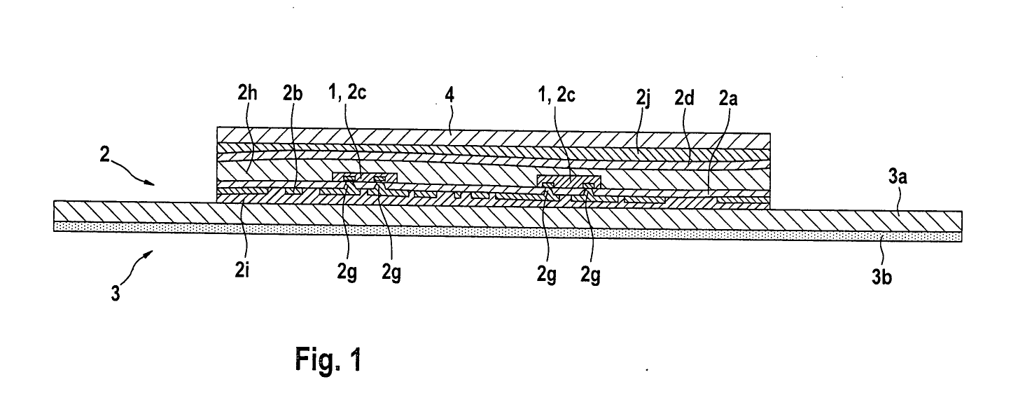

FIG. 1 shows a schematic cross section of an example embodiment of a device according to the present invention, designed as a sensor patch, for determining one or multiple body functions of a person. FIG. 1 shows that the device includes two body function sensors 1, an electronic unit 2, and a fixing means 3 for detachably fastening the device to the body of the person. The operating principle of body function sensors 1 may be based, for example, on a resistive or similar process.

FIG. 1 shows that the design of the electronic unit corresponds to a laminate-layer system design. FIG. 1 shows in particular that electronic unit 2 includes a first film 2a having a printed conductor structure 2b, two electronic components 2c, namely, body function sensors 1, situated on first film 2a and contacting printed conductor structure 2b, and a second film 2d which is laminated onto the side of first film 2a on which electronic component 2c is situated. Furthermore, FIG. 1 shows that a dielectric material 2h is situated between first film 2a and second film 2d. FIG. 1 also shows that an insulating layer 2i, 2j is applied to each of the two main surfaces of the electronic unit. FIG. 1 also shows that the fixing means includes a carrier layer 3a and an adhesive layer 3b, and that an output device 4 for cordlessly outputting data is situated opposite from adhesive layer 3b. This may involve, for example, a display or a color code.

What is claimed is:

1. A device for determining at least one body function of a person, comprising:
at least one body function sensor;
an electronic unit including: a first film having a printed conductor structure; at least one electronic component situated on the first film and contacting the printed conductor structure; and a second film laminated onto a side of the first film on which the at least one electronic component is situated; and
a fixing arrangement configured to detachably fasten the device to the body of the person.

2. The device as recited in claim 1, further comprising:
a dielectric layer situated between the first film and the second film.

3. The device as recited in claim 2, further comprising:
an insulating layer applied to at least one outwardly facing side of the electronic unit.

4. The device as recited in claim 2, further comprising:
an output device configured to cordlessly output data.

5. The device as recited in claim 4, wherein the output device is one of:
a display device;
an active transmitter for wirelessly transmitting data, wherein the active transmitter is one of an infrared transmitter or a radio transmitter; or
a passive transmitter for wirelessly transmitting data.

6. The device as recited in claim 4, wherein the output device is a display device displaying body function characteristic values.

7. The device as recited in claim 4, further comprising:
a power supply device.

8. The device as recited in claim 4, further comprising:
an evaluation device for evaluating raw data of the body function sensor.

9. The device as recited in claim 8, further comprising:
a memory device configured to store one of raw data output by the body function sensor or data at least partially evaluated by the evaluation device.

10. The device as recited in claim 9, wherein at least one of the body function sensor, the output device, the evaluation device and the memory device is at integrated into the electronic unit.

11. The device as recited in claim 9, wherein at least one of the output device, the evaluation device and the memory device is an electronic component of the electronic unit.

12. The device as recited in claim 4, wherein the fixing arrangement is configured as a patch.

13. The device as recited in claim 4, wherein the device is configured as a sensor patch.

14. A method for determining at least one body function of a person, comprising:
providing a determination device including at least one body function sensor, an output device configured to cordlessly output data, and a fixing arrangement;
measuring the at least one body function using the at least one body function sensor of the determination device, wherein the at least one body function includes at least one of heartbeat, respiration, temperature, movement of a body part, and electric body current; and
outputting by the output device data which are derived from the measured body function.
